(19) United States
(12) Patent Application Publication
(10) Pub. No.: US 2013/0204261 A1
(43) Pub. Date: Aug. 8, 2013

(54) CERAMIC CUTTING TEMPLATE

(71) Applicants: Matthias Eschle, Wannweil (DE); Roman Preuss, Kirchheim unter Teck (DE); Heinrich Wecker, Eckental (DE)

(72) Inventors: Matthias Eschle, Wannweil (DE); Roman Preuss, Kirchheim unter Teck (DE); Heinrich Wecker, Eckental (DE)

(73) Assignee: CeramTec GmbH, Plochingen (DE)

(21) Appl. No.: 13/877,718

(22) PCT Filed: Oct. 6, 2011
(86) PCT No.: PCT/EP11/67487

§ 371 Date: Apr. 4, 2013

(30) Foreign Application Priority Data
Oct. 6, 2010 (DE) 10 2010 047 473.8

(51) Int. Cl. A61B 17/17

(52) U.S. Cl. CPC A61B 17/1764
USPC 606/88

(57) ABSTRACT

The invention relates to a cutting template, to a cutting block, preferably a cutting template, and to a cutting block for use in medical technology.

Subject matter of the present invention is a cutting template and, respectively, a cutting block, preferably a cutting template and, respectively, a cutting block for use in medical technology.

Cutting templates or cutting blocks are frequently used in surgery so as to prepare or adapt the operating area, for example, when it comes to carry out implantations.

Thus, for each knee TEP implantation, a cutting template or a cutting block is fixed on the femur. In the normal case, three cuts are made with this cutting template for adapting the femur surface to the geometry of the femur component. For each cut there is a guide in the cutting template (3 or 4 cutting guides in 1 template). In this guide, the cut is carried out with a cutting instrument, normally with an oscillating saw blade. Today, saw blades and also the cutting templates are principally made from biocompatible metal alloys.

Depending on the manufacturer, the guide rails in the cutting block have a width of 1.2-1.5 mm. Due to the oscillation of the saw blade and the occurring friction between the saw blade and the guide rail, significant metallic abrasion debris from the guide rail occurs. This debris is not operatively removed or can only be removed insufficiently from the wound. Thus, in turn, this abrasion debris can become the cause of infections and can in particular result in allergic reactions in the patient. For this reason it is necessary to strictly reduce this abrasion debris, and in particular if by using a ceramic femur component, an implant reaction in the potential allergy sufferer is to be avoided.

According to the current state of knowledge, the major part of the metallic abrasion debris is caused through wear of the guide rails in the cutting template. After using a cutting template ca. 20-40 times during knee TEP implantations, the guide rails exhibit a guide gap that is increased by ca. 0.5-1.5 mm. Consequently, the guiding accuracy of the cutting template decreases significantly. Accordingly, the consequences for the surgeon are that a precisely guided cut of the saw blade is no longer possible, and alignment and evenness of the cut surfaces of the femur increasingly show variances. This results in larger gaps between the cut surfaces and the femur component. These gaps have to be filled operatively with a larger volume of bone cement than is usually the case, which can have a negative influence on the lifetime of the system.

The object underlying the present invention was to eliminate the disadvantages of the cutting templates/cutting blocks of the prior art, and in particular:
to reduce the metallic abrasion debris, wherein a reduction of the metallic abrasion debris of up to 90% with respect to the previous metal solutions shall be targeted;
to extend the lifetime of a cutting template and thus to save costs;
to reduce the allergy risk and the risk of infections.

The object according to the invention was surprisingly achieved by a cutting template/a cutting block made from ceramics (hereinafter, the terms sinter-molded body or sinter body are also used for the cutting template according to the invention/the cutting block according to the invention) with the features of the independent claims. Preferred configurations are disclosed in the sub-claims. It was surprisingly found that the solution to the upcoming objects requires sinter-molded bodies with very specific compositions.

Sinter-molded bodies that provide a solution to the present problems are so-called “yttria-stabilized tetragonal zirconia ceramics”, also called Y-TZP ceramics. Suitable according to the invention are such Y-TZP ceramics which meet the standard for medical applications.

Suitable Y-TZP ceramics contain

3 to 8% by weight of Y₂O₃, preferably 4 to 6% by weight of Y₂O₃, particularly preferred 4.5 to 5.5% by weight of Y₂O₃,

0 to 0.5% by weight of Al₂O₃, preferably 0.05 to 0.4% by weight of Al₂O₃, particularly preferred 0.1 to 0.3% by weight of Al₂O₃ and

the balance to 100% by weight of ZrO₂, wherein up to 3% by weight of HfO₂, preferably up to 2% by weight of HfO₂ can be contained in the zirconium oxide.

The monoclinic content in ZrO₂ is less than 2% by volume, preferably less than 1% by volume.

The properties of the most suitable Y-TZP ceramics exhibit a strength of from 900 to 1600 MPa, preferably of from 1000 to 1500 MPa. The grain size of the ceramic material lies in a range of <0.5 μm, preferably in a range of from 0.1 to 0.3 μm.

Preferred configurations are listed in the following table:

| | Example 1 | Example 2 | |
|---|---|---|---|
| | Value | Value | Unit |
| Yttrium oxide (Y₂O₃) | 4.9 | 5.2 | % by weight |
| Hafnium oxide (HfO₂) | 2.0 | 2.0 | % by weight |
| Aluminum oxide (Al₂O₃) | 0.1 | 0.1 | % by weight |
| Impurities | <0.1 | <0.1 | % by weight |
| Zirconium oxide (ZrO₂) | Balance to | Balance to | % by weight |
| | 100 | 100 | |

The properties of the preferably used ceramics are listed in the following tables.

### EXAMPLE 1

| | | | |
|---|---|---|---|
| Grain size | 0.15-0.2 | μm | |
| Color | ivory | | |
| Density | 6.08 | g/cm³ | DIN EN 623-2 |
| Water absorbency | 0 | % | ASTM C 373 |
| Strength (4-point bending) | 1400 | MPa | DIN ENV 843-1 |
| Weibull modulus | 10 | | DIN ENV 843-5 |
| Fracture toughness | 7.5 | MPa√m | ISO 23 146 |
| Vickers hardness (HV 0.5) | 13 | GPa | DIN ENV 843-4 |
| Young's modulus | 210 | GPa | DIN ENV 843-2 |
| Poisson's ratio | 0.3 | | DIN ENV 843-2 |
| Coefficient of thermal | | | DIN EN 821-1 |
| expansion | | | |
| 20-200° C. | 10.4 | 10⁻⁶ K⁻¹ | |
| 20-1000° C. | 11.4 | 10⁻⁶ K⁻¹ | |
| Specific heat capacity (20° C.) | 0.4 | kJ/kg K | DIN EN 821-3 |
| Thermal conductivity (20° C.) | 2.5 | W/m K | DIN EN 821-2 |

### EXAMPLE 2

| | | | |
|---|---|---|---|
| Grain size | <0.5 | μm | |
| Color | white | | DIN EN 623-2 |
| Density | 6.05 | g/cm³ | DIN EN 623-2 |
| Water absorbency | 0 | % | ASTM C 373 |
| Gas permeability | 0 | % | |
| Strength | 1050 | MPa | DIN ENV 843-1 |
| (4-point bending) | | | |
| Compressive strength | 2200 | MPa | DIN 51067 T1 |
| Weibull modulus | >10 | | DIN ENV 843-5 |
| Vickers hardness (HV 1) | 1250 | | DIN ENV 843-4 |
| Young's modulus | 210 | GPa | DIN ENV 843-2 |
| (dynamic) | | | |
| Poisson's ratio | 0.3 | | DIN ENV 843-2 |
| Coefficient of thermal | | | DIN EN 821-1 |
| expansion | | | |
| 20-200° C. | 11.1 | 10⁻⁶ K⁻¹ | |
| 20-1000° C. | 11.7 | 10⁻⁶ K⁻¹ | |
| Specific heat capacity | 0.4 | kJ/kg K | DIN EN 821-3 |
| (20° C.) | | | |
| Thermal conductivity | 2.5 | W/m K | DIN EN 821-2 |
| (20° C.) | | | |
| Thermal stress | 321 | K | |
| parameter R1 | | | |
| Dielectric constant | 29 (1 MHz) | | IEC 672-1 |
| Dielectric loss factor | 0.002 (1 GHz) | | IEC 672-1 |

In the case of the cutting templates or cutting blocks made according to the invention from Y-TZP ceramics, the metallic abrasion debris is reduced by up to 90% with respect to the previous cutting templates or cutting blocks from metal. The lifetime of the cutting templates or the cutting blocks according to the invention in use is considerably extended because only little wear occurs on the cutting templates. This reduces costs. Moreover, the allergy risk or the allergic reactions in patients and the risk of infections are reduced.

Preferably, the cutting templates according to the invention are used in medical technology, in particular during surgeries for working on a bone, preferably during knee TEP implantations.

The advantages of the ceramic cutting template according to the invention and of the ceramics of which said templates are made are:
The cutting template exhibits extremely little abrasion.
The material is biocompatible.
When the cutting template according to the invention is labeled with a laser, the label is clearly visible and legible and thus can reduce wrong handling during the use of the cutting template.
The cutting template has good tribological properties.

Producing the cutting template is carried out by means of a conventional ceramic technology.

The important process steps are:
a) Preparing the powder mixture according to the specified composition in water, use of liquefiers for avoiding sedimentation.
b) Homogenizing in the dissolver (high-speed stirrer).
c) Grinding in agitator bead mill, thereby increasing the specific surface of the powder mixture (=comminution).
d) Adding organic binders.
e) Spray drying, resulting in flowable granulate with defined properties.
f) Wetting the granulate with water.
g) Axial or isostatic pressing.
h) Machining the green part, thereby substantially forming the end contour in consideration of sinter shrinkage.
i) Pre-firing, thereby shrinking to ca. 98% of the theoretical density. The still remaining residual pores are closed from the outside.
j) Hot isostatic pressing under high temperature and high gas pressure, thereby practically reaching full final density.
k) So-called clean burn, thereby compensating the imbalance of oxygen ions in the ceramics, which imbalance is generated during hot isostatic pressing.
l) Hard machining by grinding and polishing.
m) Tempering.

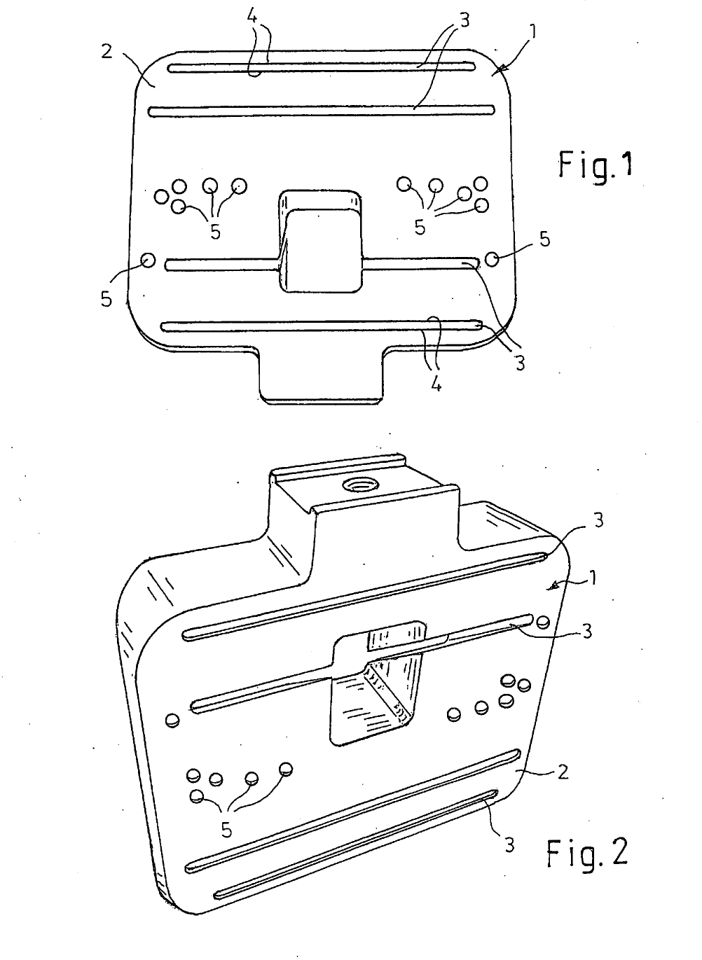

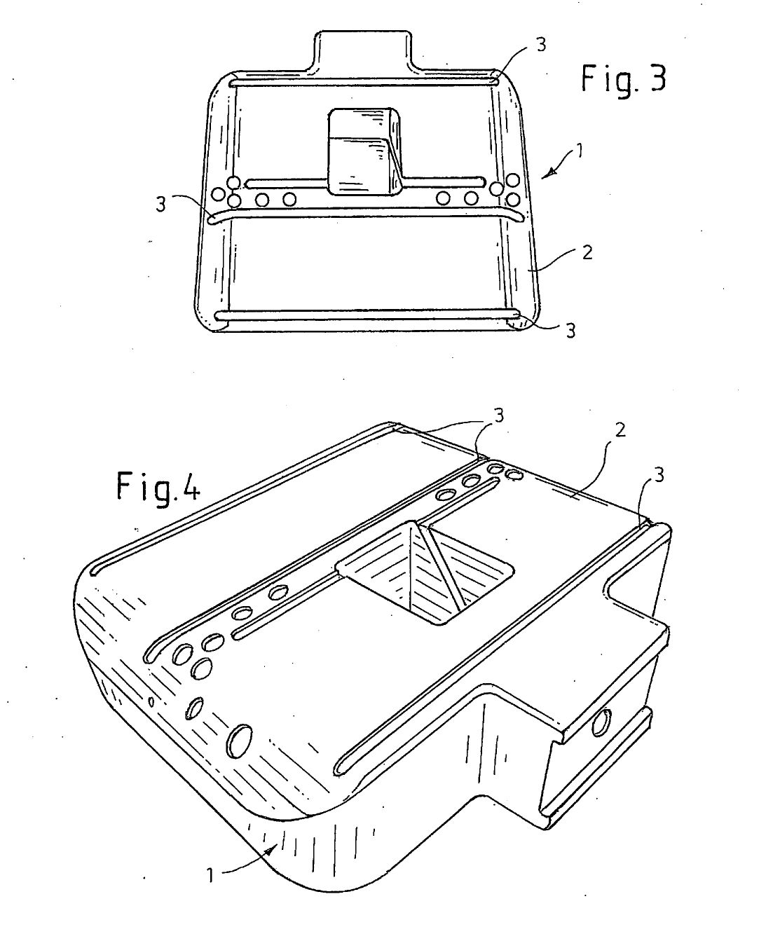

FIGS. 1 to 4 exemplary show in different views a cutting template 1 according to the invention made from ceramic for use during the implantation of an artificial knee joint. Such a cutting template 1 serves for guiding a surgical cutting instrument, for example, a saw blade or a drill.

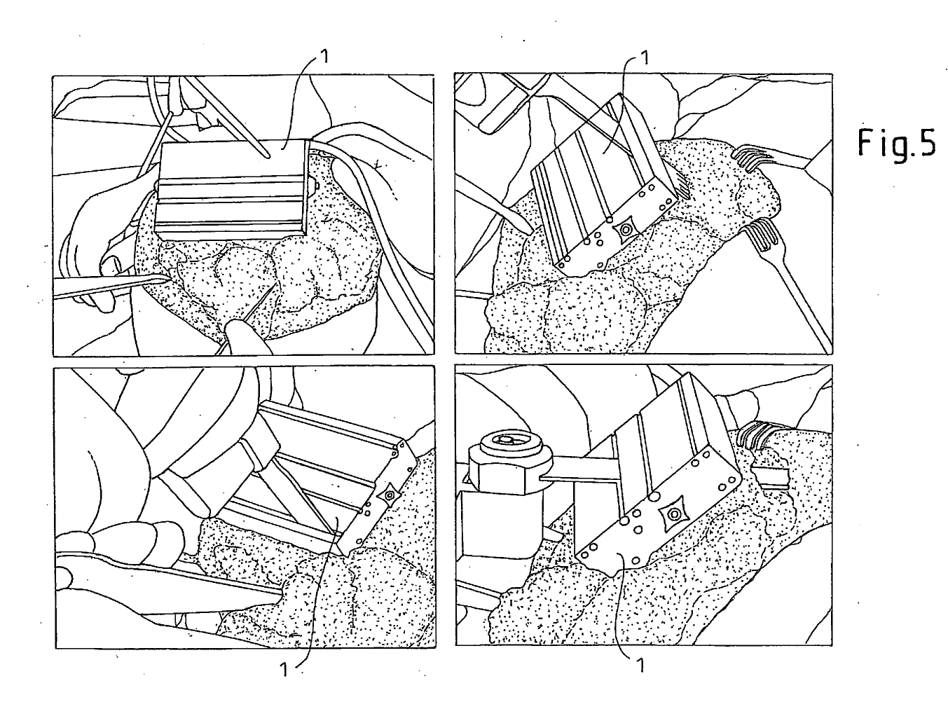

FIG. 5 is intended to illustrate the operative use of a cutting template according to the invention during the implantation of an artificial knee joint.

The cutting template shown consists of a base body 2 that is provided with slot-like recesses 3 for passing through and precisely guiding a plate-shaped cutting instrument, for example, a saw blade, wherein the slot-like recesses 3 have guide surfaces 4 that oppose each other. The cutting instrument (see FIG. 5) rests against these guide surfaces 4 during the cutting process. In the base body 2, through-holes 5 are incorporated which serve for screwing the cutting template 1 to the femur.

It is obvious for the person skilled in the art that the cutting template according to the invention can be adapted depending on the intended use. Thus, the template can also be configured in the form of a drilling template in which, for example, one or a plurality of the recesses 3 are implemented as through-hole for passing through and precisely guiding a drill as a cutting instrument.

Within the context of the present invention, the terms sinter-molded body/sinter body designate a ceramic body in the form of or for use as a cutting template or cutting block. A drilling template configured according to the invention shall also be understood as a cutting template or cutting block in the meaning of the present invention.

It follows from the above that the teaching according to the invention relates to a ceramic cutting template:
that consists of a base body 2 with one or a plurality of recesses 3 for passing through and precisely guiding a cutting instrument, wherein the ceramic material contains 3 to 8% by weight of Y₂O₃, preferably 4 to 6% by weight of Y₂O₃, particularly preferred 4.5 to 5.5% by weight of y₂O₃, 0 to 0.5% by weight of Al₂O₃, preferably 0.05 to 0.4% by weight of Al₂O₃, particularly preferred 0.1 to 0.3% by weight of Al₂O₃, and the balance to 100% by weight of ZrO₂, and wherein up to 3% by weight of HfO₂, preferably up to 2% by weight of HfO₂ can be contained in ZrO₂, and the monoclinic content in ZrO₂ is preferably less than 2% by volume, preferably less than 1% by volume.

Preferred is a ceramic cutting template in which:
the ceramic material has a strength of from 900 to 1600 MPa, preferably a strength of from 1000 to 1500 MPa;
the grain size of the ceramic material lies in a range of <0.5 μm, preferably in a range of 0.1 to 0.3 μm;
the recesses 3 for passing through and precisely guiding a cutting instrument are formed slot-like;
the recesses 3 for passing through and precisely guiding a cutting instrument are through-holes;
the slot-like recesses 3 have guiding surfaces 4 that oppose each other;
in addition, one or a plurality of through-holes 5 are incorporated in the base body 2.

1.-9. (canceled)

10. A ceramic cutting template, wherein said template comprises a base body having a recess therein for passing through and precisely guiding a cutting instrument, wherein the ceramic material comprises from 3 to 8% by weight of Y₂O₃, from 0 to 0.5% by weight of Al₂O₃, preferably 0.05 to 0.4% by weight of Al₂O₃, particularly preferred 0.1 to 0.3% by weight of Al₂O₃, and the balance to 100% by weight of ZrO₂, and wherein up to 3% by weight of HfO₂ can be contained in ZrO₂, and the monoclinic content in ZrO₂ is less than 2% by volume.

11. The ceramic cutting template according to claim 10, wherein the ceramic material has a strength of from 900 to 1600 MPa.

12. The ceramic cutting template according to claim 10, wherein the ceramic material has a grain size of <0.5 μm.

13. The ceramic cutting template according to claim 10, wherein the recess is slot-like.

14. The ceramic cutting template according to claim 10, wherein the recess is a through-hole.

15. The ceramic cutting template according to claim 13, wherein the slot-like recess comprises guiding surfaces that oppose each other.

16. The ceramic cutting template according to claim 10, wherein a through-hole is incorporated in the base body.

17. A method of performing surgery on a bone comprising utilizing the ceramic cutting template according to claim 10.

18. The method of claim 17, wherein said surgery is a knee TEP implantation.
